(19) United States
(12) Patent Application Publication
(10) Pub. No.: US 2009/0136991 A1
(43) Pub. Date: May 28, 2009

(54) Identifying Naive, Infected, or Vaccinated Mammals

(76) Inventors: Douglas E. Jones, Kelley, IA (US); Jesse Hostetter, Nevada, IA (US)

Correspondence Address: FISH & RICHARDSON P.C., PO BOX 1022, MINNEAPOLIS, MN, 55440-1022, US

(21) Appl. No.: 12/267,411

(22) Filed: Nov. 7, 2008

Related U.S. Application Data
(60) Provisional application No. 61/025,713, filed on Feb. 1, 2008.
(60) Provisional application No. 60/986,888, filed on Nov. 9, 2007.

(51) Int. Cl. C12Q 1/00; C12Q 1/06; A61M 31/00

(52) U.S. Cl. 435/39; 435/4; 604/93.01

(57) ABSTRACT

This document provides methods and materials related to assessing a mammal's immunological state. For example, methods and materials related to assessing a mammal to determine whether the mammal is immunologically naïve with respect to a pathogen, was vaccinated against that pathogen, or is infected with that pathogen are provided. This document also provides methods and materials that can be used to determine whether or not a mammal (e.g., a cow) has an early stage infection (e.g., stage 1, Johne's disease).

### CROSS-REFERENCE TO RELATED APPLICATIONS

This application claims the benefit of U.S. Provisional Application Ser. No. 61/025,713, filed Feb. 1, 2008 and U.S. Provisional Application Ser. No. 60/986,888, filed Nov. 9, 2007. The entire contents of these applications are incorporated by reference in their entirety

### BACKGROUND

1. Technical Field

This document relates to methods and materials involved in identifying naïve, infected, or vaccinated mammals. For example, this document relates to methods and materials for determining whether or not a mammal is immunologically naïve with respect to a pathogen, determining whether or not a mammal is infected with a pathogen, and determining whether or not a mammal was vaccinated against a pathogen.

2. Background Information

Mammals infected with a pathogen (e.g., a virus, a bacterium, or a fungus) can mount an immune response against that infecting pathogen. Such an immune response can include the production of antibodies. The presence of antibodies against a pathogen can indicate that the organism was exposed to that pathogen. For example, cows infected with Mycobacteria avium subspecies paratuberculosis (MAP) can contain cow antibodies that bind MAP.

MAP can cause Johne's disease, a chronic infectious disease of cattle. It is estimated that this disease causes over 200 million dollars in losses to the U.S. dairy industry annually. A significant problem associated with the control of Johne's disease in cattle is the lack of practical and accurate diagnostic assays to identify infected animals. This is especially true for infected cattle that are asymptomatic (subclinical) were failure to detect infection leads to the spread of MAP into non-infected herds and limits effectiveness of control measures in positive herds. Thus, subclinical animals represent a significant problem to the livestock industry. This is because even though they show no signs of disease, they often are very contagious. This is exacerbated by the fact that these animals likely represent the majority of the infected animals overall. Such animals are labeled Stage I cattle (where the other end of the scale, Stage IV, is end stage disease with overt clinical disease). Current diagnostic tests focus on either in vitro cell based assays (typically measurement of interferon gamma production), MAP antibody detection, or in vivo skin reactivity (skin test).

### SUMMARY

This document provides methods and materials related to assessing a mammal's immunological state. For example, this document provides methods and materials related to assessing a mammal to determine whether the mammal (1) is immunologically naïve with respect to a pathogen, (2) was vaccinated against that pathogen, or (3) is infected with that pathogen. This document also provides methods and materials that can be used to determine whether or not a mammal (e.g., a cow) has an early stage infection (e.g., stage 1, Johne's disease). Determining whether or not an animal such as a cow has an early stage infection can allow farmers to identify contagious animals. This can allow the farmer to separate animals suspected to be infected from those animals believed to be uninfected. Also, identifying immunologically naïve animals can allow farmers to vaccinate the previously uninfected population of animals as opposed to an entire herd, which can include many infected or previously infected animals.

In one embodiment, this document provides methods and materials that can be used to determine if a particular mammal received a vaccine for a particular pathogen, was infected with a naturally-occurring version of the pathogen, or is naïve with respect to the pathogen. Differentiating between vaccinated mammals and mammals infected with a naturally-occurring version of the pathogen can allow clinicians, in the case of humans, and farmers, in the case of farm animals, to determine the immunological origin of each organism's immunity to the pathogen. For example, a farmer receiving a herd of cattle can determine if the cattle of the herd received a vaccine for a particular pathogen, were infected with a naturally-occurring version of the pathogen, or are naïve with respect to the pathogen. With this information, the farmer can determine whether the herd need not be vaccinated or whether any uninfected cattle are at risk of being infected from, for example, cattle that were infected with a naturally-occurring version of the pathogen.

In general, one aspect of this document features a method for determining the immunological state of a mammal with respect to a pathogen. The method comprises, or consists essentially of: (a) administering, to the mammal, a composition comprising a matrix and an antigen preparation of the pathogen, and (b) determining whether or not the composition attracted a naïve infiltrate, wherein the presence of the naïve-infiltrate indicates that the mammal is not infected with and not vaccinated against the pathogen, and wherein the absence of the naïve-infiltrate indicates that the mammal was vaccinated against the pathogen or is infected with the pathogen. The mammal can be a bovine species. The mammal can be a cow. The pathogen can be a Mycobacteria species. The pathogen can be Mycobacteria avium. The matrix can be a gel. The matrix can be a gel that polymerizes at a temperature greater than 37° C. The matrix can be a gel that depolymerizes at a temperature below than 37° C. The matrix can comprise extracellular basement membrane matrix polypeptides. The composition can be administered subcutaneously to the mammal. The antigen preparation can comprise killed pathogens. The antigen preparation can comprise a recombinant antigen of the pathogen. The method can comprise leaving the composition within the mammal for at least 48 hours before the determining step. The method can comprise leaving the composition within the mammal for at least 72 hours before the determining step. The method can comprise leaving the composition within the mammal between about 10 and about 96 hours before the determining step. The determining step can comprise determining the number of cells within the composition or a portion of the composition. The determining step can comprise determining the level of macrophages, B cells, CD8+ T cells, dendritic cells, CD4+ T cells, gamma delta T cells, dendritic cells, or neutrophils within the composition or a portion of the composition. The determining step can comprise determining the level of IFN-gamma, IL-5, IL-6, IL-10, IL-12, IL-1b, TNF-alpha, or IL-4 within the composition or a portion of the composition. The determining step can comprise determining whether or not the composition or a portion of the composition attracted cells having the ability to produce IFN-gamma, IL-5, IL-6, IL-10, IL-12, IL-1b, TNF-alpha, or IL-4 in response to an antigen of the pathogen. The method can comprise classifying, based on information obtained from the determining step, the mammal as being (a) immunologically naïve with respect of the pathogen or (b) either infected with the pathogen or vaccinated for the pathogen.

Another aspect of this document features a delivery system for determining the immunological state of a mammal with respect to a pathogen. The delivery system comprises an injecting device configured to inject an implantation device into the mammal, a cartridge configured to engage the injecting device such that an implantation device located in the cartridge can be injected into the mammal, and a plurality of implantation devices located in the cartridge, wherein each of the plurality of implantation devices comprise a cell permeable housing and a composition located within the housing, wherein the composition comprises a matrix and an antigen preparation of the pathogen.

Another aspect of this document features a method for determining the immunological state of a mammal with respect to a pathogen. The method comprises, or consists essentially of: (a) administering, to the mammal, a composition comprising a matrix and an antigen preparation of the pathogen, and (b) determining if the composition attracted a naïve infiltrate, a vaccination infiltrate, or an infection infiltrate, wherein the presence of the naïve infiltrate indicates that the mammal is not infected with and not vaccinated against the pathogen, wherein the presence of the vaccination infiltrate indicates that the mammal was vaccinated against the pathogen, and wherein the presence of the infection infiltrate indicates that the mammal is infected with the pathogen. The mammal can be a bovine species. The mammal can be a cow. The pathogen can be a Mycobacteria species. The pathogen can be Mycobacteria avium. The matrix can be a gel. The matrix can be a gel that polymerizes at a temperature greater than 37° C. The matrix can be a gel that depolymerizes at a temperature below than 37° C. The matrix can comprise extracellular basement membrane matrix polypeptides. The composition can be administered subcutaneously to the mammal. The antigen preparation can comprise killed pathogens. The antigen preparation can comprise a recombinant antigen of the pathogen. The method can comprise leaving the composition within the mammal for at least 48 hours before the determining step. The method can comprise leaving the composition within the mammal for at least 72 hours before the determining step. The method can comprise leaving the composition within the mammal between about 10 and about 96 hours before the determining step. The determining step can comprise determining the number of cells within the composition or a portion of the composition. The determining step can comprise determining the level of macrophages, B cells, CD8⁺ T cells, dendritic cells, CD4⁺ T cells, gamma delta T cells, dendritic cells, or neutrophils within the composition or a portion of the composition. The determining step can comprise determining the level of IFN-gamma, IL-5, IL-6, IL-10, IL-12, IL-1b, TNF-alpha, or IL-4 within the composition or a portion of the composition. The determining step can comprise determining whether or not the composition or a portion of the composition attracted cells having the ability to produce IFN-gamma, IL-5, IL-6, IL-10, IL-12, IL-1b, TNF-alpha, or IL-4 in response to an antigen of the pathogen. The method can comprise classifying, based on information obtained from the determining step, the mammal as being immunologically naïve with respect of the pathogen, as being infected with the pathogen, or as being vaccinated for the pathogen.

Another aspect of this document features a kit for determining the immunological state of a mammal with respect to a pathogen. The kit comprises, or consists essentially of: (a) an implantation device comprising a cell permeable housing and a composition located within the housing, wherein the composition comprises a matrix and an antigen preparation of the pathogen, and (b) a detection device comprising an antibody for detecting a cytokine or an inflammatory cell. The mammal can be a bovine species. The mammal can be a cow. The pathogen can be a Mycobacteria species. The pathogen can be Mycobacteria avium. The matrix can be a gel. The matrix can be a gel that polymerizes at a temperature greater than 37° C. The matrix can be a gel that depolymerizes at a temperature below than 37° C. The matrix can comprise extracellular basement membrane matrix polypeptides. The antigen preparation can comprise killed pathogens. The antigen preparation can comprise a recombinant antigen of the pathogen. The antibody can be capable of detecting macrophages, B cells, CD8⁺ T cells, dendritic cells, CD4⁺ T cells, gamma delta T cells, dendritic cells, or neutrophils. The antibody can be capable of detecting IFN-gamma, IL-5, IL-6, IL-10, IL-12, IL-1b, IL-4, or TNF-alpha. The cell permeable housing can comprise a PET membrane or perfluorosulfonic acid polymer. The cell permeable housing can comprise a cylindrical shape. The cell permeable housing can comprise a hollow needle shape.

Another aspect of this document features a method for distinguishing between vaccinated and infected mammals. The method comprises, or consists essentially of: (a) administering, to a mammal, a composition comprising a matrix and an antigen preparation of a pathogen, and (b) determining if the composition attracted a vaccination infiltrate or an infection infiltrate, wherein the presence of the vaccination-infiltrate indicates that the mammal was vaccinated against the pathogen, and wherein the presence of the infection-infiltrate indicates that the mammal is infected with the pathogen. The mammal can be a bovine species. The mammal can be a cow. The pathogen can be a Mycobacteria species. The pathogen can be Mycobacteria avium. The matrix can be a gel. The matrix can be a gel that polymerizes at a temperature greater than 37° C. The matrix can be a gel that depolymerizes at a temperature below than 37° C. The matrix can comprise extracellular basement membrane matrix polypeptides. The composition can be administered subcutaneously to the mammal. The antigen preparation can comprise killed pathogens. The antigen preparation can comprise a recombinant antigen of the pathogen. The method can comprise leaving the composition within the mammal for at least 48 hours before the determining step. The method can comprise leaving the composition within the mammal for at least 72 hours before the determining step. The method can comprise leaving the composition within the mammal between about 10 and about 96 hours before the determining step. The determining step can comprise determining the number of cells within the composition or a portion of the composition. The determining step can comprise determining the level of macrophages, B cells, CD8⁺ T cells, dendritic cells, CD4⁺ T cells, gamma delta T cells, dendritic cells, or neutrophils within the composition or a portion of the composition. The determining step can comprise determining the level of IFN-gamma, IL-5, IL-6, IL-10, IL-12, IL-1b, IL-4, or TNF-alpha within the composition or a portion of the composition. The determining step can comprise determining whether or not the composition or a portion of the composition attracted cells having the ability to produce IFN-gamma, IL-5, IL-6, IL-10, IL-12, IL-1b, IL-4, or TNF-alpha in response to an antigen of the pathogen. The method can comprise classifying, based on information obtained from the determining step, the mammal as being infected with the pathogen or as being vaccinated for the pathogen.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DESCRIPTION OF DRAWINGS

FIG. 1A is a photograph of gross swelling 72 hours after injection of Matrigel containing killed MAP (10⁵; MAP+gel) into the tail head of a calf experimentally infected with MAP. FIG. 1B is a photograph of a histological stain (H&E staining, magnification=400×) of a gel containing no MAP (gel alone) 72 hours after inoculation into a calf experimentally infected with MAP. FIG. 1C is a photograph of a histological stain (H&E staining, magnification=400×) of a gel containing killed MAP (10⁵) 72 hours after inoculation into a calf experimentally infected with MAP.

FIG. 2 is a graph plotting the number of cells harvested from either Matrigel containing killed MAP (10⁵) or gel alone injected into calves vaccinated for MAP or experimentally infected with MAP.

FIG. 3 contains graphs plotting flow cytometry results of an analysis of cellular constituents from a single Matrigel containing killed MAP (10⁵) recovered from a calf experimentally infected with MAP. FIG. 3A is a graph of flow cytometry results for macrophages using anti-CD14 and anti-CD68 antibodies. FIG. 3B is a graph of flow cytometry results for B cells using an anti-B cell antibody (B-B4 antibody). FIG. 3C is a graph of flow cytometry results for CD8 T cells using anti-CD8 antibodies. FIG. 3D is a graph of flow cytometry results for CD4/CD25 positive T cells using anti-CD4 and anti-CD25 antibodies. FIG. 3E is a graph of flow cytometry results for gamma delta T cells using anti-gamma delta TCR antibodies. FIG. 3F is a graph of flow cytometry results for dendritic cells using anti-CD11c and anti-DEC 205 antibodies.

FIG. 4 is a graph plotting the percent of each indicated cell type recovered in Matrigel containing killed MAP (10⁵) from either calves vaccinated for MAP or experimentally infected with MAP.

FIG. 5 is a bar graph plotting the lesion score for sites in either uninfected mice (UN) or mice infected with L. major (LM) that received Matrigel alone (NONE), Matrigel containing killed Leishmania major (LM), or Matrigel containing killed L. amazonensis (LA). The first two letters designate the infection status of the mouse (LM=L. major, UN=uninfected), and the last letters indicated the challenge antigen (LM=L. major, LA=L. amazonensis, NONE=no antigen). Data represents three replications of the experiment.

FIG. 6 contains photographs of the morphology of injection sites of L. major-infected mice injected with Matrigel alone (LM/NONE), Matrigel containing killed L. major (LM/LM), or Matrigel containing killed L. amazonensis (LM/LA).

FIG. 7 is a bar graph plotting the level (pg/mL) of the indicated cytokines recovered from injection sites of L. major-infected mice injected with Matrigel alone (LM/NONE), Matrigel containing killed L. major (LM/LM), or Matrigel containing killed L. amazonensis (LM/LA).

FIG. 8 contains data for MAP-infected and uninfected animals. One set of experiments was performed. There was a unique cellular profile for test sites between MAP infected and non-infected animals. This correlates to peripheral blood gamma stimulation tests. Local cytokine profiles can be evaluated.

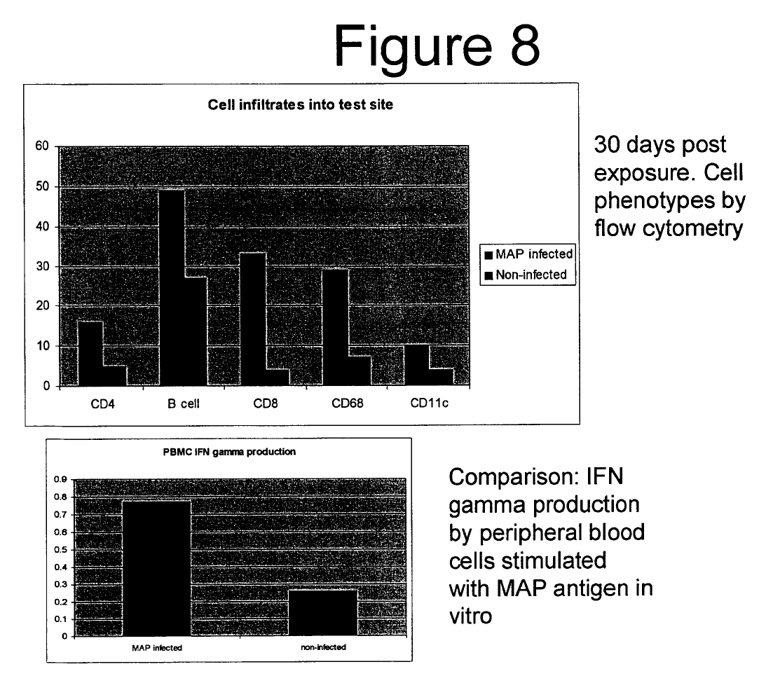

FIG. 9 contains photographs of typical gross (a) and microscopic (b) appearances of Matrigel alone (No Map) after 48 hours in the subcutis of a MAP infected calf and photographs of typical gross (c) and microscopic (d) appearances of Matrigel+10⁵ live Map K10 after 48 hours in the subcutis of a MAP infected calf. The Matrigel with MAP antigen (c) exhibited marked cloudiness, which corresponded to an intense inflammatory cell infiltrate into the Matrigel (d). This cellular infiltrate is characteristic of a delayed type hypersensitivy reaction.

FIG. 10 is a graph plotting the mean amount of IFN-gamma (pg/mL) within Matrigel's with MAP antigen (MAP) or without MAP antigen (NO MAP) recovered from the indicated calves. CON indicates control calves that were not inoculated with MAP or vaccinated. INF indicates calves infected with MAP. VAC indicates calves vaccinated for MAP. The vaccinated animals produced the highest amount of IFN-gamma, MAP infected animals produced an intermediate level, and the control animals produced little IFN-gamma. The results represent three replications of the experiment with 9 animals per group.

FIG. 11 is a bar graph plotting the cell number index for each indicated surface marker for a vaccinated animal (VAC), an infected animal (INF), and a non-infected control (CON). The cell number index is the ratio of cells that migrated into the Matrigel containing MAP antigen divided by the number of cells that migrated into the Matrigel without MAP antigen. The X axis are the different inflammatory cell phenotypes evaluated. Each animal exhibited a unique cellular profile.

FIGS. 12A and 12B are cross-sectional views of exemplary implantation devices.

FIGS. 13A, 13B, and 13C are cross-sectional views of exemplary implantation devices.

FIG. 14 is top view of exemplary injection device.

FIGS. 15A and 15B are top views of exemplary cartridge assemblies.

### DETAILED DESCRIPTION

This document provides methods and materials related to assessing mammals to determine whether or not the mammals were exposed to a pathogen via, for example, a vaccination against the pathogen (e.g., vaccination with a vaccine of recombinant polypeptides of the pathogen or a vaccine of attenuated pathogen) or a pathogen infection. For example, this document provides methods and materials for assessing an mammal's immunological state with respect to a pathogen. The methods and materials provided herein can be used to assess any type of mammal including, without limitation, mice, rats, dogs, cats, horses, cows, pigs, goats, monkeys, and humans.

As described herein, an antigen preparation can be administered to a mammal to be tested, and the cellular response within the mammal that is provoked by the administered antigen preparation can be assessed to determine the immunological state of the mammal with respect to one or more pathogens for which antigens are present within the antigen preparation. An antigen preparation can contain one antigen (e.g., one recombinant polypeptide of a pathogen) of one pathogen. In some cases, an antigen preparation can contain many antigens of a pathogen. For example, an antigen preparation can contain killed bacterial pathogens. In some cases, an antigen preparation can contain antigens for more than one pathogen (e.g., two, three, four, five, six, or more pathogens). For example, an antigen preparation can contain killed bacterial pathogens and killed fungal pathogens. A pathogen can be a bacterial pathogen, a fungal pathogen, a viral pathogen, or a protozoal pathogen. Examples of bacterial pathogens include, without limitation, Mycobacteria species (e.g., M. avium or M. tuberculosis), Salmonella species, Borellia species, Francisella species, Coxiella species, and Brucella species. Examples of fungal pathogens include, without limitation, Histoplasmosis species, Blastomycosis species, coccidiomycosis species, and Aspergillus species. Examples of viral pathogens include, without limitation, influenza viruses, papilloma viruses, herpesviruses, morbilliviruses, and corona. Examples of protozoal pathogens include, without limitation, Leishmania species (e.g., L. major), toxoplasma species, plasmodium species, sarcocystis species, and amoeba species.

An antigen preparation can be administered alone or in combination with a matrix. For example, an antigen preparation of M. avium can be mixed with a matrix to form a composition that can be administered to a mammal to be tested. A matrix can be a gel, a liquid, or a solid. In some cases, a matrix can contain laminin, heparan sulfate proteoglycans, collagen (e.g., collagen Type IV), entactin, growth factors, cytokines, and/or chemokines. For example, a matrix can contain IL-12. In some cases, a matrix can have the ability to polymerize or solidify at temperatures greater than 30° C. (e.g., greater than 31, 32, 33, 34, 35, 36, 37, 38, 39, 40, 41, 42, 43, 44, or 45° C.). In some cases, a matrix can have the ability to de-polymerize or liquefy at temperatures less than 40° C. (e.g., less than 39, 38, 37, 36, 35, 34, 33, 32, 31, or 30° C.). For example, a matrix can have the ability to polymerize or solidify at temperatures greater than 37° C. and can have the ability to de-polymerize or liquefy at temperatures less than 37° C. Examples of matrix compositions that can be used as described herein include, without limitation, Matrigel® (BD Biosciences; San Jose, Calif.), pentoblock co-polymers, and Pluronic® (block copolymers based on ethylene oxide and propylene oxide; BASF).

In some cases, a composition containing a matrix and an antigen preparation can contain particles (e.g., microparticles or nanoparticles) containing one or more capture antibodies. The capture antibodies can be used to capture polypeptides such as cytokine or chemokine polypeptides. For example, a composition can include a matrix, an antigen preparation, and microparticles coated with anti-IFN-γ antibodies.

Any appropriate amount of an antigen preparation can be administered to a mammal to be tested. In general, between about 0.1 μg and 5 mg (e.g., between about 0.1 μg and 2.5 mg, between about 0.1 μg and 2 mg, between about 0.1 μg and 1.5 mg, between about 0.1 μg and 1.0 mg, between about 0.1 μg and 750 μg, between about 0.1 μg and 500 μg, between about 0.1 μg and 250 μg, between about 0.1 μg and 100 μg, between about 0.5 μg and 5 mg, between about 0.75 μg and 5 mg, between about 1 μg and 5 mg, between about 2.5 μg and 5 mg, between about 5 μg and 5 mg, between about 10 μg and 5 mg, between about 100 μg and 5 mg, between about 1 μg and 1 mg, or between about 5 μg and 500 μg) of an antigen preparation can be administered to a mammal. When administered in combination with a matrix, the combination can be administered such that between about 50 μL and 2.5 mL (e.g., between about 75 μL and 2.5 mL, 100 μL and 2.5 mL, 150 μL and 2.5 mL, 200 μL and 2.5 mL, 250 μL and 2.5 mL, 300 μL and 2.5 mL, 50 μL and 2 mL, 50 μL and 1.5 mL, 50 μL and 1.0 mL, 50 μL and 750 μL, 50 μL and 500 μL, 50 μL and 250 μL, or 100 μL and 1 mL) is delivered to the mammal. In some cases, between about 0.1 and 5 percent (e.g., between about 0.2 and 5 percent, between about 0.3 and 5 percent, between about 0.4 and 5 percent, between about 0.1 and 4 percent, between about 0.1 and 3 percent, between about 0.1 and 2 percent, between about 0.1 and 1 percent, or between about 0.1 and 0.7 percent) of a composition containing a matrix and an antigen preparation can be the antigen preparation. For example, at least about 0.5 percent of a composition containing a matrix and an antigen preparation can be the antigen preparation. In some cases, about 20 μg of an antigen preparation can be mixed with about 5 mg (about 10 mg/mL) of Matrigel to form a composition that can be administered to a mammal such that the mammal receives about 500 μL of the composition.

Any appropriate route of administration can be used to administer an antigen preparation to a mammal to be tested. For example, an antigen preparation provided herein can be injected under the skin of a mammal. Other routes of administration include, without limitation, subcutaneous injections, intramuscular injections, and intradermal injections.

Once administered to a mammal, a composition containing an antigen preparation can be allowed to remain in the mammal for at least about two hours (e.g., at least about 24, 36, 48, 60, 72, 84, or 96 hours). In some cases, a composition containing an antigen preparation can be administered to a mammal and allowed to remain in the mammal for between about two hours and about 10 days. After remaining within the mammal for a period of time, a sample from the administration site (e.g., injection site) can be removed from the mammal and assessed to determine the type of cellular infiltrate, if any, that was attracted to the administered composition.

In some cases, the type of cellular infiltrate can be determined based on the number of cells present within a sample. For example, the presence of few, if any, cells (e.g., inflammatory cells) can indicate that the cellular infiltrate is that of a naïve mammal. The presence of a substantial number of cells (e.g., greater than about 2.8×10⁵ cells per μL) can indicate that the cellular infiltrate is that of a mammal infected with a low to moderately pathogenic pathogen. The presence of a moderate number of cells (e.g., between about 4×10³ and about 2.8×10⁵ cells per μL) can indicate that the cellular infiltrate is that of a vaccinated mammal or a mammal infected with a highly pathogenic pathogen. Any method can be used to determine the number of cells within a sample. For example, cells can be identified based on their forward scatter/side scatter profiles and counted using flow cytometry.

In some cases, the type of cellular infiltrate can be determined based a cellular index determined for a sample. A cellular index can be used as a measure of cellular infiltrates into the gels. The cellular index can be a ratio of the cells present in the gel containing antigen divided by the number of cells within the gels containing gel alone (recovered from the same animal). A cellular index of >1.25 for the measured cell types (e.g., CD8, CD4, B cells, CD14, DEC 205, Gamma delta T cells) can indicate that an animal is either infected or vaccinated. A cellular index of <1.25 for the measured cell types (e.g., CD8, CD4, B cells, CD14, DEC 205, Gamma delta T cells) can indicate that an animal is naïve for the tested pathogen. In some cases, a cellular index can be used to discriminate vaccinated animals from infected animals. For example, a cellular index above about 1.5 for CD4 cells or CD14 cells can indicate that an animal has been vaccinated against the tested pathogen.

In some cases, the type of cellular infiltrate can be determined based on the types of cells present within a sample. For example, the presence of an infiltrate that has greater than 30 percent B cells, greater than 10 percent CD8+ T cells, or greater than 20 percent gamma delta T cells can indicate that the cellular infiltrate is that of an infected mammal. The presence of an infiltrate that has between 10 and 40 percent macrophages and either less than 30 percent B cell, less than 10 percent CD8+ T cells, or less than 20 percent gamma delta T cells can indicate that the cellular infiltrate is that of a vaccinated mammal. Any method can be used to determine the type of cells within a sample. For example, cells can be counted by flow cytometry using appropriate antibodies (e.g., anti-CD4 antibodies, anti-CD8 antibodies, anti-CD68 antibodies, anti-CD14 antibodies, anti-CD25 antibodies, anti-WC1 antibodies, anti-CD62 antibodies, anti-CD45RO antibodies, or anti-CD45RA antibodies). In some cases, a cellular infiltrate can be assessed for the presence of activated cells (e.g., CD45RA+ cells) and/or memory cells (e.g., CD45RO+ cells). The presence of such cells can indicate that the cellular infiltrate is that of an infected or vaccinated mammal.

In some cases, the type of cellular infiltrate can be determined based on the types of polypeptides (e.g., cytokines or chemokines) present within a sample or the types of polypeptides produced by the cellular infiltrate. For example, the presence of an infiltrate that has moderate to substantial levels of IFN-γ or IL-6 or cells producing moderate to substantial levels (e.g., greater than 100 pg/mL, 200 pg/mL, 300 pg/mL, 400 pg/mL, 500 pg/mL, 600 pg/mL, 700 pg/mL or more) of IFN-γ or IL-6 in response to the antigen preparation can indicate that the cellular infiltrate is that of an infected mammal. The presence of an infiltrate that has moderate levels of IL-12 or cells producing moderate levels of IL-12 in response to the antigen preparation can indicate that the cellular infiltrate is that of a vaccinated mammal. In some cases, the presence of an infiltrate that lacks moderate to substantial levels of IFN-γ or IL-6 or lacks cells producing moderate to substantial levels of IFN-γ or IL-6 in response to the antigen preparation can indicate that the cellular infiltrate is that of a vaccinated mammal. Any appropriate method can be used to determine the types of polypeptides present within a sample or the types of polypeptides produced by the cellular infiltrate. For example, the level of a particular polypeptide can be determined by enzyme-linked immunoassays (ELISAs) using appropriate antibodies (e.g., anti-IFN-γ antibodies, anti-IL-5 antibodies, anti-IL-6 antibodies, anti-IL-12 antibodies, anti-IL-10 antibodies, anti-IL-1a antibodies, anti-IL-1b antibodies, anti-IL-2 antibodies, or anti-IL-4 antibodies).

Any appropriate method can be used to determine the level of a particular polypeptide based on the formation of antibody-polypeptide complexes. For example, an indicator molecule having binding affinity for the antibody-polypeptide complex can be used to detect an antibody-polypeptide complex. As used herein, an “indicator molecule” is any molecule that allows the presence of a given polypeptide, antibody, or antibody-polypeptide complex to be visualized, either with the naked eye or an appropriate instrument. Typically, the indicator molecule is an secondary antibody having binding affinity for a primary antibody used to bind a polypeptide of interest. Indicator molecules can be detected either directly or indirectly by standard methodologies. See, e.g., Current Protocols in Immunology, Chapters 2 and 8, Coligan et al., (eds.), John Wiley & Sons (1996). For direct detection, the indicator molecule can be labeled with a radioisotope, fluorochrome, other non-radioactive label, or any other suitable chromophore. For indirect detection methods, enzymes such as horseradish peroxidase (HRP) and alkaline phosphatase (AP) can be attached to the indicator molecule, and the presence of the antibody-polypeptide complex can be detected using standard assays for HRP or AP. In some cases, the indicator molecule can be attached to avidin or streptavidin, and the presence of the antibody-polypeptide complex can be detected with biotin conjugated to, for example, a fluorochrome, or vice versa. Thus, assay formats for detecting antibody-polypeptide complexes can include ELISA such as competitive ELISAs, radioimmunoassays (RIA), fluorescence assays, chemiluminescent assays, immunoblot assays (e.g., Western blots), particulate-based assays, and other known techniques. In some cases, antibody-polypeptide complexes are formed in solution. Such complexes can be detected by immunoprecipitation. See, e.g., Short Protocols in Molecular Biology, Chapter 10, Section VI, Ausubel et al., (eds.), Green Publishing Associates and John Wiley & Sons (1992).

In some cases, aptamers can be used to identify and quantify parameters within a sample (e.g., a gel) including lymphocyte subsets, activated dendritic cells, macrophages, and secreted polypeptides including chemokines and cytokines. Aptamers can be polypeptides or oligonucleotides that bind with high affinity and specificity to a specific target molecule and can distinguish between closely related targets.

In some cases, the lymphocyte population of a cellular infiltrate can yield information with respect to identification of early infected animals (e.g., stage 1 infections). For example, the presence of dendritic cells, macrophages, and lymphocytes within a cellular infiltrate can indicate that an animal has been exposed to MAP and is in the early stages of infection. In addition, lymphocytes subsets can be used to determine if the exposed animal has mounted a protective immune response and is “cured” or has a suboptimal immune response and will likely progress to shedding and clinical disease. For example, the presence of CD25⁺ T cells, CD4⁺ T cells, CD8⁺ T cells, and gamma delta T cells within a cellular infiltrate can indicate that an animal has been exposed and is mounting a protective cell mediated immune response.

As described herein, the methods and materials provided herein can be used to determine a mammal's immunological state with respect to a pathogen. Such methods and materials can be used to determine an immunological state in any mammal. For example, the immunological state of a pig, dog, cat, sheep, cow, horse, goat, monkey, or human can be determined using the methods and materials provided herein. In some cases, the methods and materials provided herein can be used to determine a non-mammalian animal's immunological state with respect to a pathogen. For example, the immunological state of a bird (e.g., chicken, turkey, or duck) can be determined using the methods and materials provided herein. In addition, an animal's immunological state (e.g., a mammalian or non-mammalian animal's immunological state) with respect to any pathogen can be determined. For example, a cow's immunological state with respect to a bacterial pathogen such as M. avium can be determined.

In some cases, the methods and materials provided herein can be used to determine whether a mammal's immunological state is such that (1) the mammal received a vaccine version of a pathogen, (2) the mammal was infected with a naturally-occurring version of the pathogen, or (3) the mammal is immunologically naive with respect to the pathogen. In some cases, the methods and materials provided herein can be used to differentiate between mammals having either an immunological state such that (1) the mammal received a vaccine version of a pathogen or (2) the mammal was infected with a naturally-occurring version of the pathogen.

In some cases, to improve the ease of use, a gel matrix can be contained within a housing or membrane material that is permeable to cell infiltration into the gel. Components that make up such a membrane can include perfluorosulfonic acid polymer (see, e.g., U.S. Pat. No. 5,624,537) or micron pore size PET membranes (e.g., between about 30 and 110 micron pore size PET membranes, between about 40 and 100 micron pore size PET membranes, about 70 micron pore size PET membranes, or the like). In some cases, the pore sizes can be about 8 microns.

The housing material that contains the gel matrix can advantageously be configured such that one or more of the housings can be installed in a delivery device for efficient, high-speed, delivery of one or more matrices, each into an individual animal.

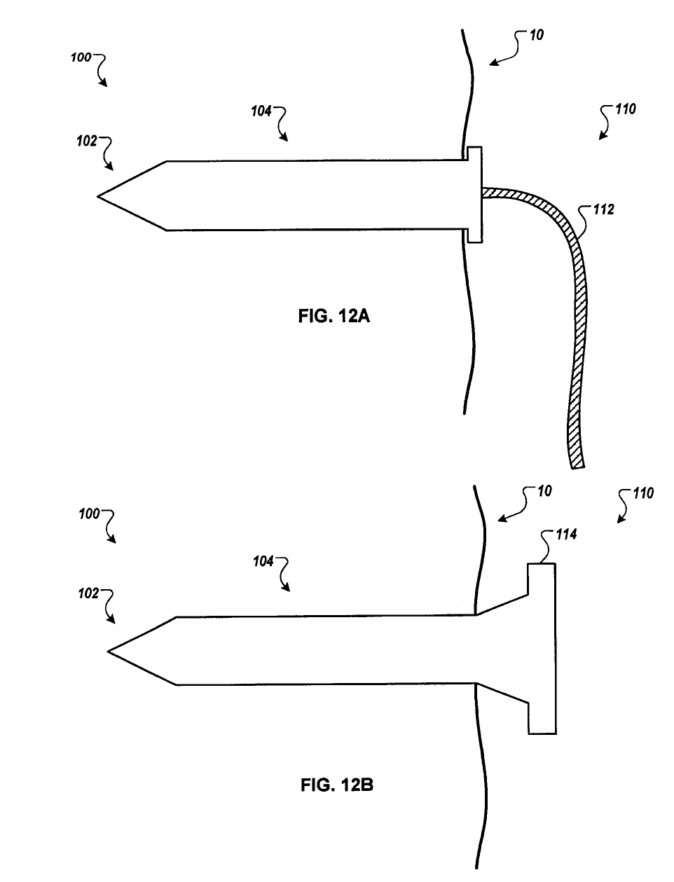

Referring now to FIGS. 12A-B, an implantation device 100 can be used for the injection of an antigen preparation under the skin 10 of an animal. At least a portion of the implantation device 100 can be removed at a later time for analysis (e.g., to determine the type and/or number of cells that have infiltrated into the implantation device 100). For example, the implantation device 100 can be delivered under the skin of an animal and allowed to remain in the animal (e.g., for at least about 2, 24, 36, 48, 60, 72, 84, or 240 hours). After remaining in the animal for a period of time, at least a portion of the implantation device can be removed to determine the type and amount of cellular infiltrate present within the implantation device 100.

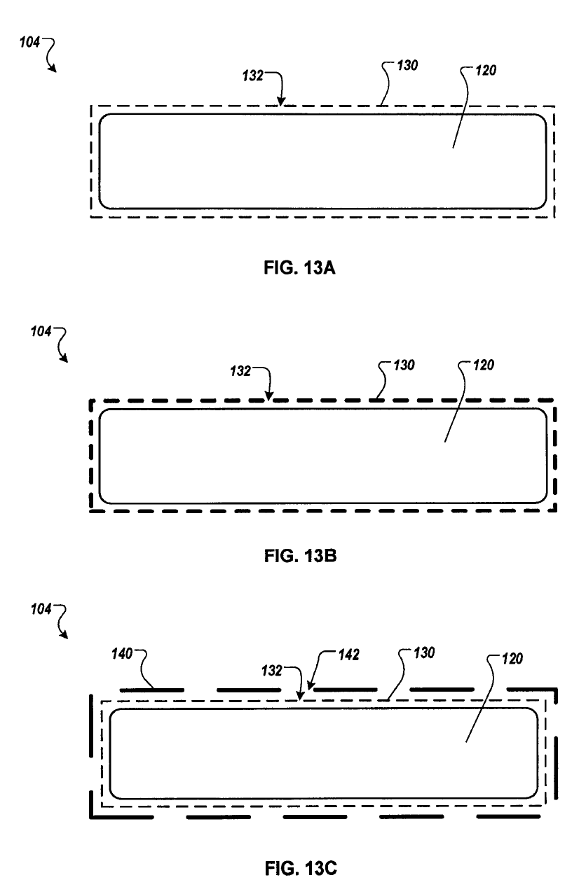

In some cases, implantation device 100 can include an insertion portion 102 that tapers down to, for example, a point or tip (e.g., a rounded tip) to facilitate insertion of the implantation device under the skin of an animal. The implantation device can include a matrix portion 104 that can contain, for example, a housing, a gel matrix, and the like, to be delivered under the skin of an animal. The matrix portion 104 is described in greater detail in connection with FIGS. 13A-13B.

In some cases, implantation device 100 can include a removal mechanism 110. In FIG. 12A, the removal mechanism can include a removal string 112 coupled to the back end of the implant (e.g., the end of the implant closest to the surface of the skin). In use, after the implantation device 100 has remained in an animal for a period of time, the implantation device 100 can be removed by pulling on string 112, thus dislodging implantation device 100 from the tissue of the animal. String 112 can continue to be pulled until the implantation device 100 is removed from the animal. In some cases, such as that depicted in FIG. 12B, removal mechanism 110 can include a graspable removal plug 114 coupled to the back of the implantation device 100. In these cases, after implantation device 100 has remained in an animal for a period of time, implantation device 100 can be removed by grasping and pulling on removal plug 114 until implantation device 100 is removed from the tissue of the animal. When removed, implantation device 100 can be analyzed to determine the type and/or number of cells present within implantation device 100.

Referring now to FIGS. 13A-C, matrix portion 104 of implantation device 100 (see FIGS. 12A-B), for injection under the skin of an animal, can include a gel matrix 120 (e.g., including an antigen preparation) contained within a first porous housing 130. The first porous housing 130 can contain the matrix 120, while allowing the infiltration of cells (e.g., cells attracted to antigen preparation such as inflammatory cells). In some cases, such as those depicted in FIGS. 12A-B, first porous housing 130 can be strong and stiff enough to maintain substantially its original shape after injection under the skin of an animal (e.g., as part of implantation device 100), but be porous enough for the infiltration of cells. For example, as depicted in FIG. 13A, porous housing 130 can include perfluorosulfonic acid polymer or micron pore size PET membranes with a pore size of between about 30 and 110 microns, between about 40 and 100 microns, about 70 microns, or the like. In the example depicted in FIG. 13B, housing 130 can include materials, such as metal, that can provide greater stiffness and strength to the porous housing 130, while maintaining desired pore sizes and total pore surface area. In some cases, as depicted in FIG. 12C, implantation device 100 can include more than one porous housing. For example, implantation device 100 can include gel matrix 120, first porous housing 130, and a second porous housing 140. In this example, first porous housing 130 and second porous housing 140 can cumulatively provide the strength and stiffness needed to maintain implantation device 100 in substantially its original shape after injection under the skin of an animal, while providing desired pore size and total pore area. In some cases, first porous housing 130 can include a material that is less strong and has pores 132 of the desired sizes (e.g., between about 30 and 110 microns, between about 40 and 100 microns, or about 70 microns), while second porous housing 140 can include a stronger material (e.g., metal) to provide added strength and stiffness, and pores 142 that are larger than pores 132. In some cases, second porous housing 140 can be replaced with a frame configured to provide appropriate strength and stiffness to first porous housing 130. Such a frame can be constructed of metal or other appropriate materials.

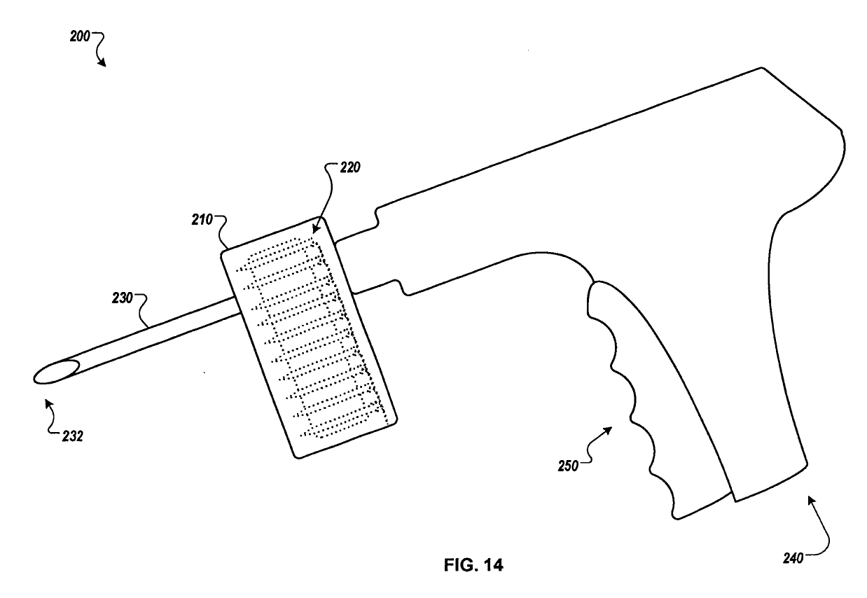

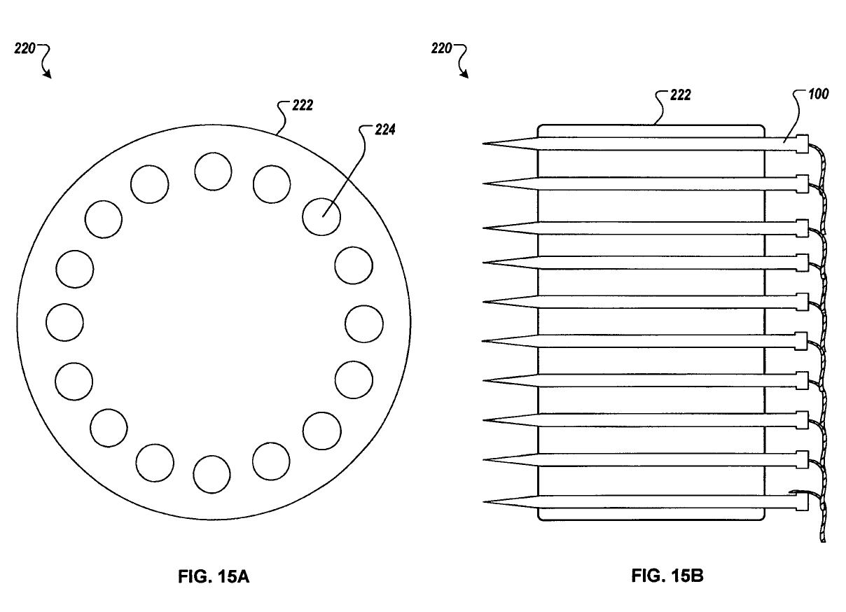

Referring now to FIG. 14, implantation devices, such as implantation devices 100 depicted in FIGS. 12A-B, can be injected under the skin of an animal using an injection device 200. Injection device 200 can include a cartridge housing 210 that can contain a cartridge assembly 220 (described in greater detail in connection with FIGS. 15A-B) and an injection shaft 230. In use, an operator can grasp injection device 200 by handle 240 and position tip 232 of injection shaft 230 against the skin of an animal. The operator can penetrate the skin of the animal with tip 232 of injection shaft 230 by applying pressure via handle 240. Once tip 232 has penetrated the skin of the animal, trigger 250 can be actuated, causing one of the implantation devices to be ejected from injection device 200, via shaft 230, such that the implantation device is placed under the skin of the animal. Injection device 200 can be withdrawn and reused to place additional implants into the same animal or to place implants into different animals (e.g., each animal of heard).

Referring now FIGS. 15A-B, the cartridge assembly can include a cartridge 222 for holding one or more implantation devices 100 for use in injection device 200. One or more implantation devices 100 can be inserted into cartridge 222 at orifices 224. When implantation device 100 has been inserted, the cartridge assembly (including cartridge 222 and implantation devices 100) can be installed in cartridge housing 210 of injection device 200 (see FIG. 14) for use in injecting one or more of the implantation devices under the skin of animals.

The invention will be further described in the following examples, which do not limit the scope of the invention described in the claims.

### EXAMPLES

### Example 1

### Diagnostic Assay for MAP Infection by Rapid Quantification of In Vivo Immune Responses to MAP

To obtain MAP-infected calves, four week old weaned calves were inoculated with 109 live MAP CFU subcutaneously in the right side of the neck. To obtain MAP-vaccinated calves, four week old weaned calves were inoculated with a commercially available MAP vaccination in the same location according to the manufacturer's specifications (Mycopar, Fort Dodge Laboratories). Calves were housed in appropriate facilities for the course of the experiments.

A combination of MAP antigens (10⁵ killed MAP bacteria) incorporated into Matrigel (BD Biosciences; San Jose, Calif.) (MAP+Gel) was subcutaneously injected into the tailhead of MAP-vaccinated calves or MAP-infected calves. Briefly, 500 μL of cooled Matrigel was mixed thoroughly with 10⁵ killed MAP (ATCC strain 19698) bacteria. This material was injected subcutaneously into the right side of the tailhead in animals that were infected or vaccinated 60 days earlier. After 72 hours, the injection site was removed by excisional biopsy. The recovered material was divided into two equal portions. Half went into 10% neutral buffered formalin for histopathological analysis, and the second have was cooled to induce depolymerization.

Cells were isolated from the gel by simply de-polymerizing the gel by cooling it to below 37° C. followed by centrifugation (200 g for 15 minutes). The cells that infiltrated into the Matrigel were washed and stained using primary antibodies for CD4, CD8, WC.1, CD44, CD25, B-cell marker, CD11c, CD14, and CD40 (VMRD Pullman Wash.) followed by fluorescently conjugated secondary antibodies (VMRD, Pullman Wash.). The cells also were stained for surface immunoglobulin subtypes: IgM, IgA, IgG1, and IgG2a. Intracellular staining for IFN-γ, TNF-α, and IL-10 was completed. These parameters were measured using flow cytometry.

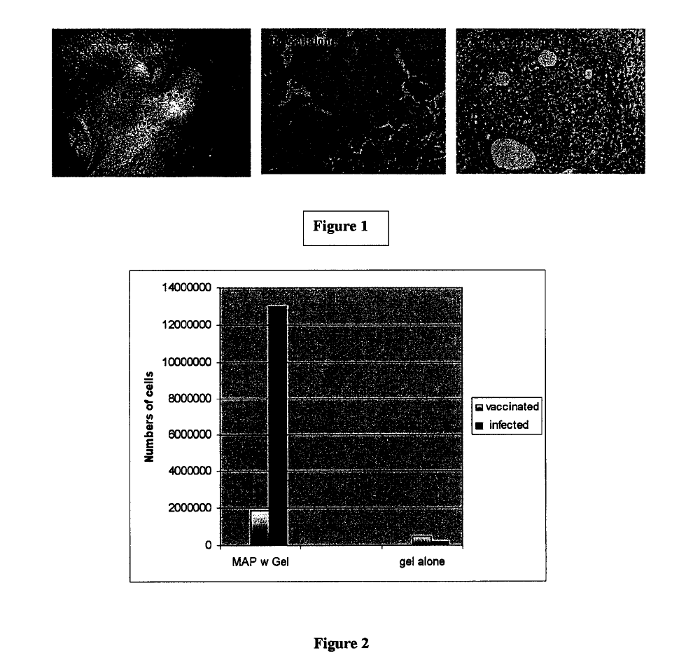

Before removal of the gel from the calves, the inoculation sites appeared to be similar to the standard skin test reaction. Also, no difference was observed between the swelling in the vaccinated animals and the experimentally infected animals. A representative photograph of an injection site for a gel containing MAP injected into a calf infected with MAP is provided (FIG. 1A). These results demonstrate that the gross appearance of the Matrigel injection site was similar between the vaccinated and infected animals.

Histologically, the gel alone, whether injected into a MAP-vaccinated or MAP-infected calf for 72 hours, did not attract a strong inflammatory response (FIG. 1B). A representative photograph of histological staining of an injection site of a gel containing MAP injected into a calf infected with MAP is provided (FIG. 1C). The gel containing killed MAP injected into an MAP-infected calf for 72 hours attracted a strong inflammatory response (FIG. 1C), while the same gel injected into an MAP-vaccinated calf for 72 hours attracted a slight, but noticeable less inflammatory response. These results demonstrate that by itself, the gel does not induce substantial inflammatory cell infiltration and that MAP antigen is required. These results also demonstrate that the degree of inflammatory cell infiltration differs between MAP-vaccinated or MAP-infected animals.

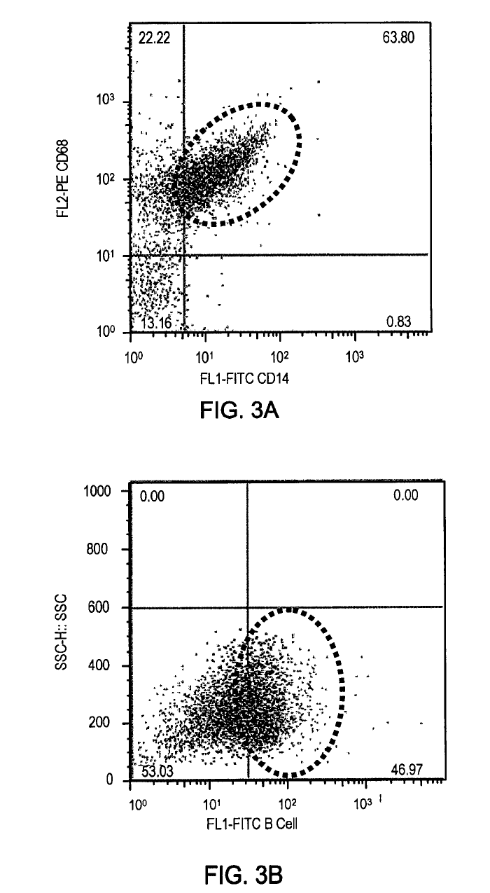

The cellular density within gels was determined by flow cytometry. Cell numbers were determined from forward scatter and side scatter dot blots using flow cytometric measurement of the depolymerized gels. The absolute numbers of all inflammatory cells that infiltrated into the gel in MAP-vaccinated or experimentally MAP-infected calves were determined and compared to numbers of cells that infiltrated into gels lacking MAP bacteria. The numbers of cells responding to MAP antigen was found to be the highest for the experimentally MAP-infected animals (FIG. 2).

These results demonstrate that sufficient numbers of cells infiltrate into the gel with 72 hours for detection by flow cytometry. These results also demonstrate that a clear difference exists between vaccinated calves and calves experimentally infected with MAP, even though the injection sites of vaccinated calves and experimentally infected calves presented similar measurements with respect to overall observable swelling. Thus, vaccinated animals can be distinguished from infected animals based on cellular infiltration into the gel despite similar sizes of the skin swelling.

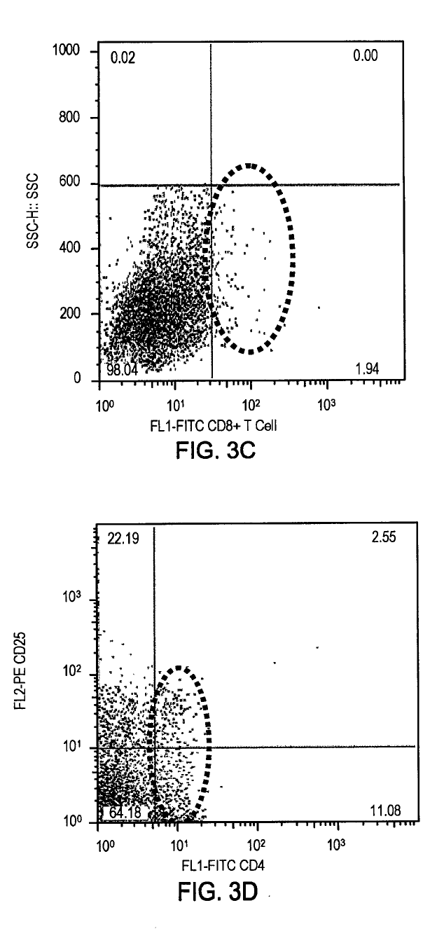

The cellular composition within the gel containing killed MAP was determined. A panel of markers for lymphocytes and antigen presenting cells was selected. The highest percentage of cells were macrophages followed by B lymphocytes (FIG. 3). Other cell types including T cell subsets were present at lower percentages (e.g., gamma delta T cells, CD8 T cells, CD25/CD4 dual positive T cells, and dendritic cells (CD11/DEC205 dual positive cells)). In some cases, these were at low numbers, yet were well within the detectable limits of the assay. These results demonstrate that sufficient cellular infiltration into the gel exists to identify and quantify multiple cellular phenotypes that have responded to the antigen after 72 hours.

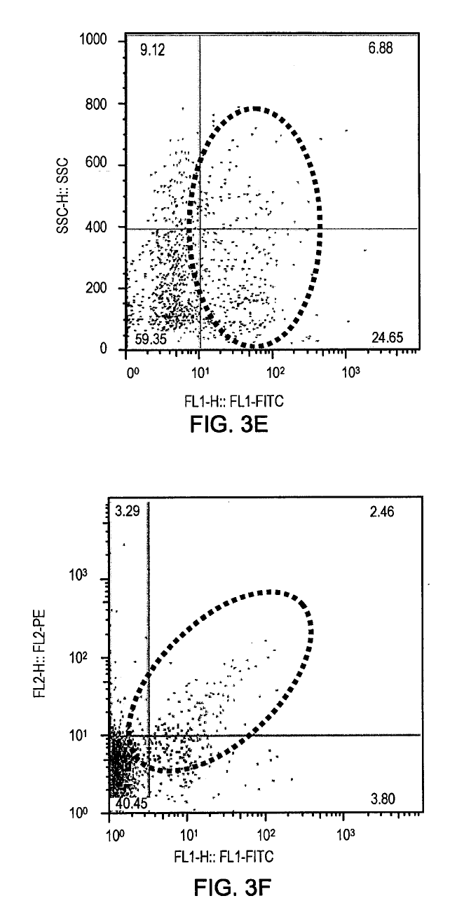

Cell profiles were determined to further evaluate the ability to discriminate vaccinated animals from infected animals. Accurately identifying MAP-vaccinated cattle can be a problem within the industry in places where vaccination is routinely used as the vaccinates can be positive by most of the assays used to detect MAP infection, including a standard skin test. The cell profiles of the infiltrating cells differed between the MAP-vaccinated and MAP-infected calves (FIG. 4). The differences were most apparent for B cells, CD8 T cells, and gamma delta T cells (FIG. 4) as well as for the overall number of responding cell types (FIG. 2). These results demonstrate that vaccinated and infected animals have unique cellular profiles that can be determined from cells that infiltrate gels containing antigen.

### Example 2

### Diagnostic Assay for Leishmania

Mice were infected with Leishmania major or left as uninfected controls. After four months, mice were inoculated with Matrigel containing L. major (20 μg killed MHOM/IL/80 Freidlin), Matrigel containing L. amazonensis (20 μg killed MHOM/BR/OO/LTB0016), or Matrigel alone. 72 hours post inoculation, the gel site was recovered, processed, and stained with hematoxylin and eosin. Sections were scored histologically for degree of cellular infiltration and organization of the infiltrate. In addition, photo micrographs were taken at 200× magnification of hematoxylin and eosin stained sections from formalin fixed paraffin embedded tissues.

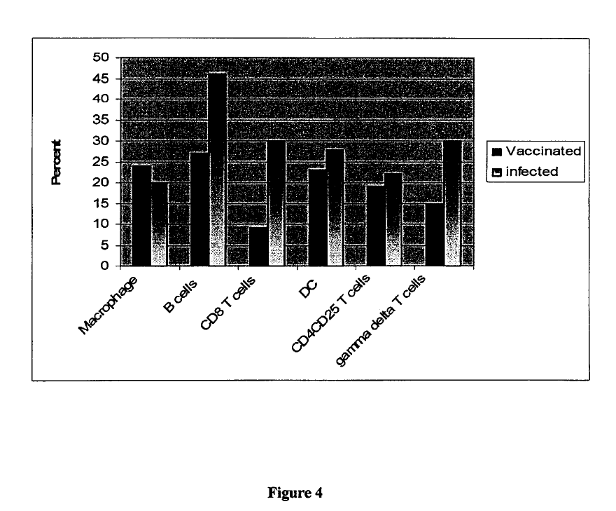

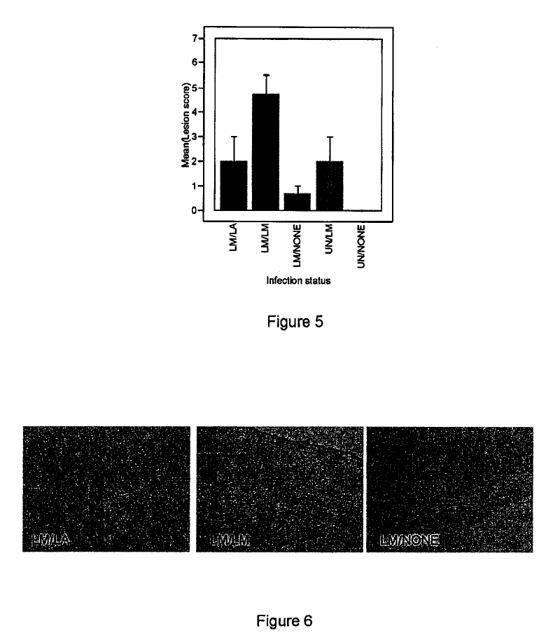

Mice injected with a gel containing the same species as the infecting pathogen exhibited the highest scores for cellular infiltration into the gel test site (FIG. 5). Cells infiltrating into gels containing the corresponding antigen exhibited characteristics consistent with a delayed type hypersensitivity response, while gels without antigen evoked little inflammation (FIG. 6). These images demonstrate the existence of cellular infiltration and organization into the gels after 72 hours in vivo in mice infected with L. major. The most extensive inflammatory cell infiltrate was into the gel that contained L. major. The infiltration included neutrophils, macrophages, and lymphocytes and was consistent with the components of a delayed type hypersensitivity response. These results demonstrate that antigen loaded gels can be placed into an infected animal, which elicits a detectable antigen specific cellular infiltration.

Gels recovered from mice were incubated in 96 well plates for 72 hours at 37° C. without further antigen stimulation. Following incubation, microbeads coated with capture antibody for IFN-gamma, IL-5, IL-6, IL-10, and IL-12 were added. Cytokine concentrations were determined by ELISA using a cytometric array platform (Luminex).

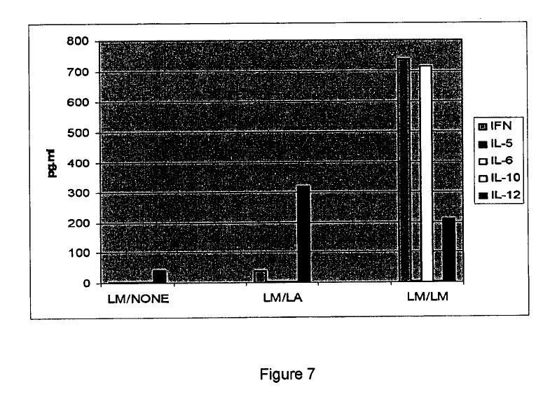

Cytokine profiles for the selected cytokines were unique for mice infected with L. major when stimulated with L. major antigens in the gel (FIG. 7). These mice exhibited high IFN-γ and IL-6, while L. major infected mice challenged with L. amazonensis antigen had high to moderate levels of IL-12, but low IFN-γ and IL-6. These results demonstrate that distinct cytokine profiles are induced by a pathogen and can be detected using the methods and materials provided herein. These results also demonstrate that cytokine profiles can be used to identify infected animals.

### Example 3

### Assessing Cellular Infiltrates in Calves

Nine 6 week old calves were divided into three groups. The first group received MAP vaccine (Mycopar—Fort Dodge Animal Health) subcutaneously, the second group was inoculated with 1×10⁹ live MAP organisms in the subcutis of the neck, and the third group were left as negative controls.

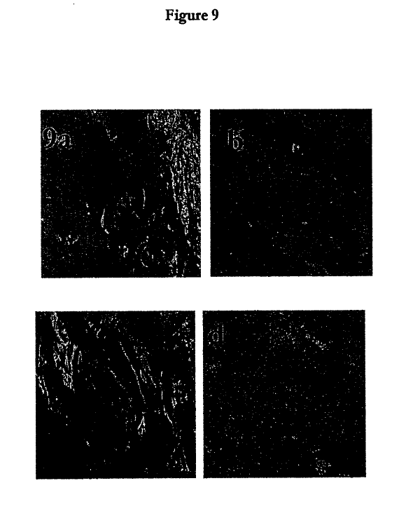

30 days post inoculation, two injections of Matrigel were injected subcutaneously into the skin of the neck on the opposite side of the previous inoculations. Each Matrigel injection contained 500 μL of Matrigel. One gel contained 10⁵ live MAP organisms, and the other contained no MAP antigen (negative control). Photographs of the appearance of the Matrigel within subcutis of the animal were taken (FIG. 9). The Matrigel with no antigen was clear, while the Matrigel with antigen was cloudy (FIG. 9). This cloudiness corresponded to a marked cellular infiltration into the gel, which is significant because it is this environment that is directly sampled in this technique for cellular infiltrate and protein production.

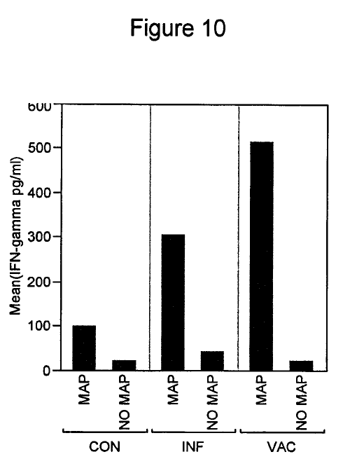

The gels were surgically removed after 48 hours and cooled to de-polymerize. After centrifugation, the supernatant was incubated with Luminex beads coated with appropriate IFN-gamma capture antibodies then secondary antibodies. Recombinant bovine IFN-gamma was used to establish a standard curve. IFN-gamma levels were then measured by a flow cytometric technique. The concentration of IFN-gamma in each treatment group was determined (FIG. 10). These results demonstrate that there is little production of IFN-gamma in the Matrigel that does not contain MAP antigen. These results also demonstrate that animals that were vaccinated have a very high level of IFN-gamma with infected animals having an intermediate concentration. Both vaccinates and infected animals had higher levels of IFN-gamma than the negative control animal. This is significant because it demonstrates that cutoff values for IFN-gamma concentration in this system can be used to discriminate between the following groups of animals: infected animals, vaccinated animals, and non-infected animals.

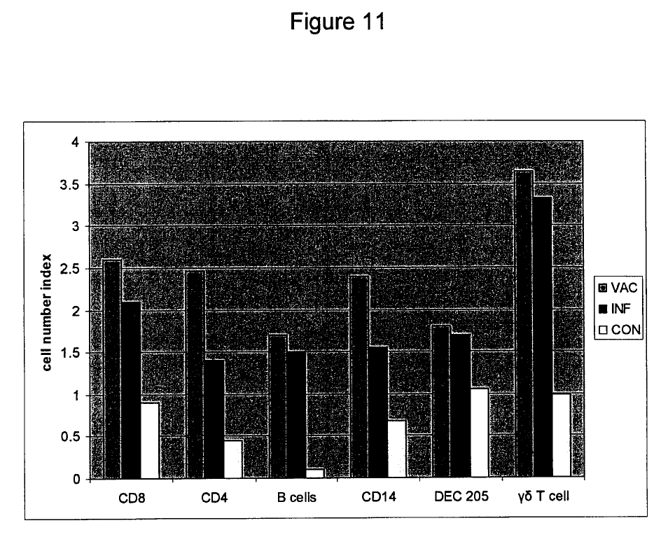

The cells recovered from the gel were stained for a panel of surface marker antigens including: CD8, CD4, Gamma Delta T cell receptor, B cell receptor, CD14, and CD205. Commercially available bovine specific primary antibodies were used followed by appropriately fluorescently tagged secondary antibodies. Cellular phenotypes were then determined by flow cytometry. The cellular profiles from a vaccinated calf (VAC), an infected calf (INF), and a control calf (CON) were determined (FIG. 11). These revealed the presence of a typical cellular profile for 30 days post inoculation with MAP. In this data set, the cellular index was the absolute number of cells that migrated into the Matrigel plus antigen divided by the absolute number of cells that migrated into the Matrigel alone (on that animal). Therefore, this was a true representation of that animal's response to the mycobacterial antigens present. These results demonstrate that the profiles of all three animals are unique. These profiles can be used to establish reference limits for each parameter and taken together can provide a fingerprint of an infected, non-infected, and vaccinated animals.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for determining the immunological state of a mammal with respect to a pathogen, wherein said method comprises:
(a) administering, to said mammal, a composition comprising a matrix and an antigen preparation of said pathogen, and
(b) determining whether or not said composition attracted a naïve infiltrate,
wherein the presence of said naïve-infiltrate indicates that said mammal is not infected with and not vaccinated against said pathogen, and wherein the absence of said naïve-infiltrate indicates that said mammal was vaccinated against said pathogen or is infected with said pathogen.

2. The method of claim 1, wherein said mammal is a bovine species.

3. The method of claim 1, wherein said pathogen is a Mycobacteria species.

4. The method of claim 1, wherein said pathogen is Mycobacteria avium.

5. The method of claim 1, wherein said matrix is a gel.

6. The method of claim 1, wherein said matrix is a gel that polymerizes at a temperature greater than 37° C.

7. The method of claim 1, wherein said matrix is a gel that depolymerizes at a temperature below than 37° C.

8. The method of claim 1, wherein said composition is administered subcutaneously to said mammal.

9. The method of claim 1, wherein said antigen preparation comprises killed pathogens.

10. The method of claim 1, wherein said method comprises leaving said composition within said mammal for at least 48 hours before said determining step.

11. The method of claim 1, wherein said determining step comprises determining the number of cells within said composition or a portion of said composition.

12. The method of claim 1, wherein said determining step comprises determining the level of macrophages, B cells, CD8+ T cells, dendritic cells, CD4+ T cells, gamma delta T cells, dendritic cells, or neutrophils within said composition or a portion of said composition.

13. The method of claim 1, wherein said method comprises classifying, based on information obtained from said determining step, said mammal as being (a) immunologically naïve with respect of said pathogen or (b) either infected with said pathogen or vaccinated for said pathogen.

14. A delivery system for determining the immunological state of a mammal with respect to a pathogen, wherein said delivery system comprises an injecting device configured to inject an implantation device into said mammal, a cartridge configured to engage said injecting device such that an implantation device located in said cartridge can be injected into said mammal, and a plurality of implantation devices located in said cartridge, wherein each of said plurality of implantation devices comprise a cell permeable housing and a composition located within said housing, wherein said composition comprises a matrix and an antigen preparation of said pathogen.

15. A kit for determining the immunological state of a mammal with respect to a pathogen, wherein said kit comprises:
(a) an implantation device comprising a cell permeable housing and a composition located within said housing, wherein said composition comprises a matrix and an antigen preparation of said pathogen, and
(b) a detection device comprising an antibody for detecting a cytokine or an inflammatory cell.

16. The kit of claim 15, wherein said mammal is a bovine species.

17. The kit of claim 15, wherein said pathogen is a Mycobacteria species.

18. The kit of claim 15, wherein said pathogen is Mycobacteria avium.

19. The kit of claim 15, wherein said matrix is a gel.

20. The kit of claim 15, wherein said matrix is a gel that polymerizes at a temperature greater than 37° C.
